(19) United States
(12) Patent Application Publication
(10) Pub. No.: US 2012/0142937 A1
(43) Pub. Date: Jun. 7, 2012

(54) METHOD FOR COUPLING HALOGEN-SUBSTITUTED AROMATIC COMPOUNDS WITH ORGANIC COMPOUNDS COMPRISING TRIALKYLSILYL-SUBSTITUTED HETEROATOMS

(75) Inventors: Andreas Kreipl, Hamburg (DE); Nicolas Boege, Hamburg (DE); Werner R. Thiel, Kaiserslautern (DE)

(73) Assignee: Zylum Beteiligungsgesellschaft mbH & Co. Patente II KG, Schonefeld/Waltersdorf (DE)

(21) Appl. No.: 13/256,516

(22) PCT Filed: Mar. 16, 2010
(86) PCT No.: PCT/EP2010/053413

§ 371 Date: Nov. 14, 2011

(30) Foreign Application Priority Data
Mar. 16, 2009 (EP) 09155232.3

(51) Int. Cl. C07D 233/61; C07D 231/12; C07F 9/50; C07C 253/30; C07C 41/14; C07D 233/72; C07D 295/155; C07D 295/073

(52) U.S. Cl. 548/321.1; 548/343.5; 548/577; 548/377.1; 558/424; 568/584; 564/15

(57) ABSTRACT

A process is provided for the preparation of aryl-heteroatom-bridged compounds by reacting a halogen-substituted aromatic compound with a trialkylsilyl-substituted heteroatom-containing organic compound.

### CROSS-REFERENCE TO RELATED APPLICATIONS

This application is a Section 371 of International Application No. PCT/EP2010/053413, filed Mar. 16, 2010, which was published in the German language on Sep. 23, 2010, under International Publication No. WO 2010/106078 A1 and the disclosure of which is incorporated herein by reference.

### BACKGROUND OF THE INVENTION

The invention relates to a process for preparing aryl-heteroatom-bridged compounds. Aryl-heteroatom-bridged compounds in the sense of the present invention are in particular understood to be compounds in which an aryl residue is bound directly to a heteroatom, in particular N or O, which in turn is part of a heteroatom-containing organic residue.

Aryl-heteroatom-bridged compounds are valuable starting materials for organic synthesis. They are particularly valuable precursors for the synthesis of active pharmaceutical ingredients, as well as of pesticides and herbicides.

Previous syntheses of such compounds are based on a combinatorial strategy, and require a suitable coupling method with sometimes complex protecting group strategy or transition metal-catalyzed steps. The use of protecting groups is associated with the disadvantage that the incorporation of the protecting group prior to coupling and subsequent splitting off of the protection group are associated with additional reaction steps, which increases the complexity of the process control significantly and thus leads to higher costs and greater susceptibility of the synthesis route to disruption. Furthermore, a protecting group strategy needs to be tailored to the respective functional group to be protected as well as to the other substances present in the reaction mixture, so that there is no universally applicable process scheme, which would be usable for various reactions of this type.

There is widespread use of transition metal catalysts in organic synthesis. Such transition metal catalysts are, however, associated with the disadvantage that they are often quite expensive on the one hand and, on the other hand, can contaminate the reaction product with toxic heavy metal compounds. Especially when using the compounds as starting material for the synthesis of active pharmaceutical ingredients, this is a serious drawback, requiring elaborate and costly purification steps.

Thus, for example, the Buchwald-Hartwig cross-coupling reaction is a palladium-catalyzed synthesis of aryl amine, which proceeds according to the following reaction scheme:

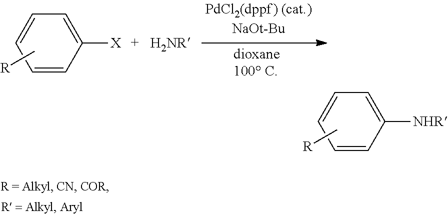

Known examples from the recent literature for reactions of this type are:

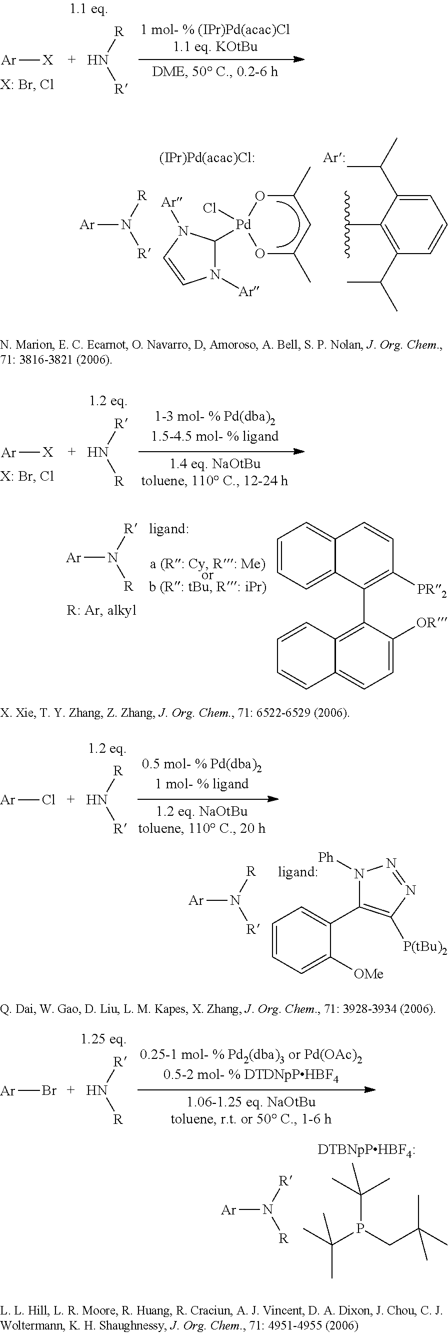

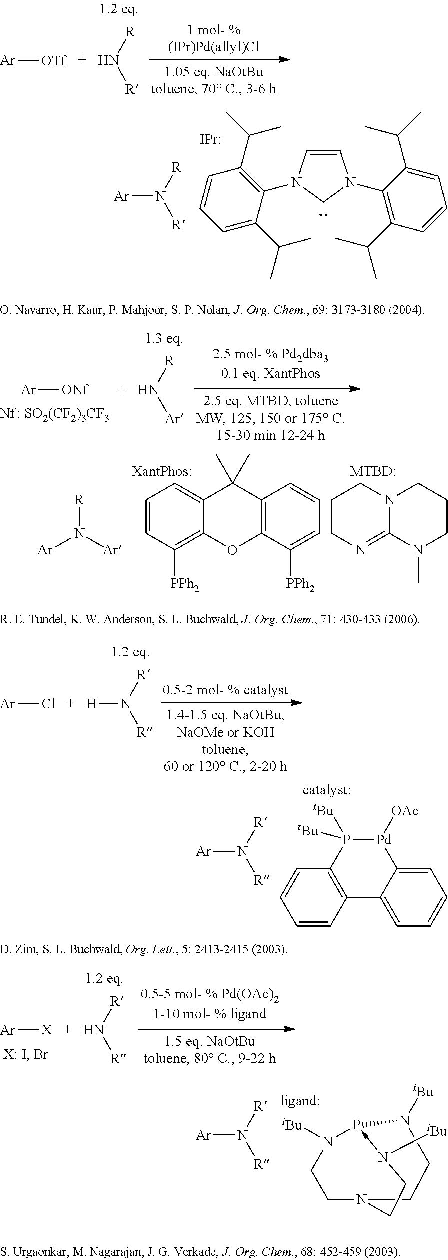

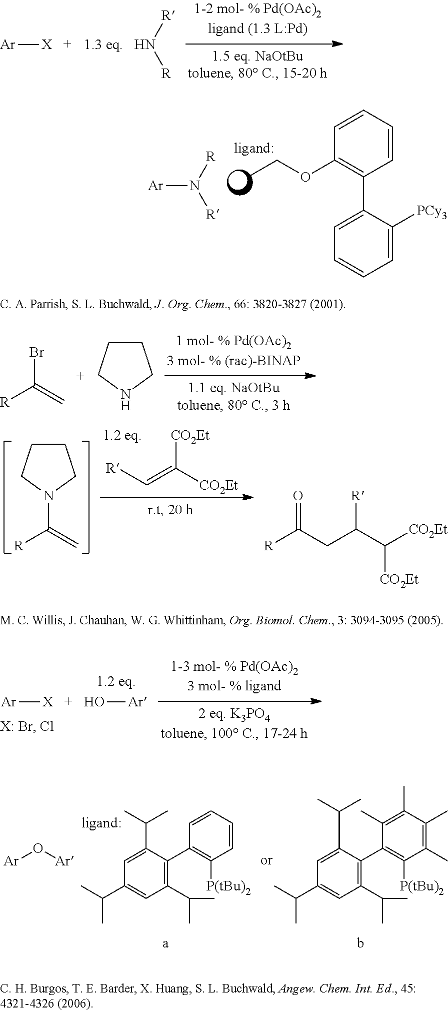

In sum, it should be noted that the previous processes of preparing aryl-heteroatom-bridged compounds by cross-coupling with the use of protecting groups require an elaborate process control and thus lead to high costs. Moreover, these methods usually give relatively low yields. Transition metal-catalyzed reactions are associated with the disadvantage of contaminating the finished product with toxic heavy metals.

The use of trialkylsilyl groups as substituents in organic synthesis has gained a certain importance in recent years. Typically, trialkylsilyl groups are employed therein as protecting groups, which, during the course of a multistep synthesis, should prevent reaction of the existing functional groups with reagents that are used. Some syntheses have been described in which trialkylsilyl substituents are directly involved in the reaction.

An example of a reaction of this type is the Hiyama coupling, in which a palladium-catalyzed CC bond formation between aryl, alkenyl or alkyl halides or pseudohalides and organosilanes takes place according to the following reaction scheme:

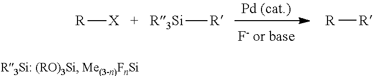

Examples from the recent literature for reactions of this type are:

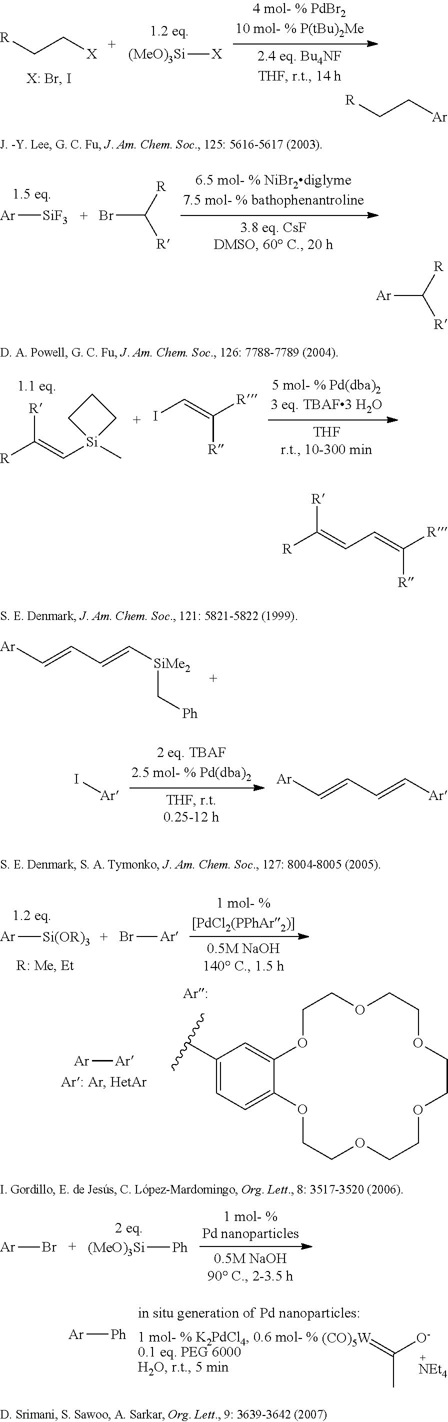

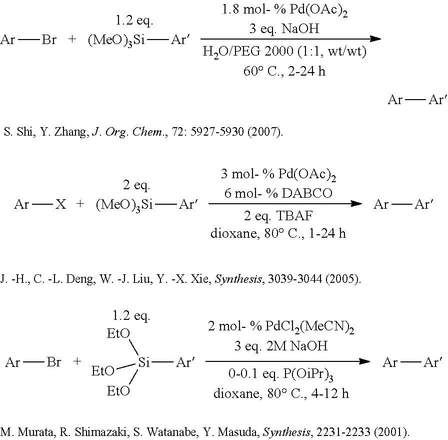

German Patent Publication DE 37 38 276 describes a process for preparing polyarylene sulfides, in which polyarylene sulfides are prepared by melt condensation from halogenated arylthiosilanes. The reaction takes place in the presence of at least one alkali and/or alkaline earth metal or ammonium halide as the catalyst.

U.S. Patent Application Publication No. 2008-0042127 describes the coupling of bridged silyl compounds with perfluorinated aromatic compounds using fluoride ions for the preparation of compounds, which are suitable for the production of electronic components.

U.S. Pat. No. 5,523,384 describes the preparation of polyether ketones using silanolates and copper catalysts.

As described above, all known processes for the production of aryl-heteroatom bridged compounds have serious disadvantages, especially in terms of complexity of reaction control, achievable yields, and under some circumstances the presence of toxic end product contamination.

### BRIEF SUMMARY OF THE INVENTION

In view of the above, the object of the present invention is to provide a process for producing aryl-heteroatom-bridged compounds, which compared to the previous processes is simpler, faster and cheaper to perform, leads to high yields, and avoids the toxic heavy metal contamination of the end products.

The above-mentioned object is achieved by a process which is characterized in that a halogen-substituted aromatic compound is reacted with a trialkylsilyl-substituted heteroatom-containing organic compound.

Surprisingly, it has been shown that, by this synthesis strategy, previously difficultly available aryl-heteroatom-bridged compounds may be obtained in high yield in a one-step synthesis. The reaction proceeds under mild conditions without the use of transition metal-containing catalysts.

In a preferred embodiment, a mono- or di-halogen-substituted aromatic compound is used as the halogen-substituted aromatic compound.

Particularly good results are achieved if a fluorine-substituted aromatic compound is used as the halogen-substituted aromatic compound.

Furthermore, in a preferred embodiment a trimethylsilyl-substituted heteroatom-containing organic compound is used as a trialkylsilyl-substituted heteroatom-containing organic compound.

The process is basically suitable for the preparation of aryl-heteroatom-bridged compounds of various structures. By using readily available trialkylsilyl-activated heteroatom-containing organic compounds, a large number of different aryl-heteroatom-bridged compounds become available in high yields in a single reaction step. In particular, the trialkylsilyl-substituted heteroatom-containing compound is a compound in which the trialkylsilyl group(s) is (are) bound as leaving group(s) to the heteroatom(s) to be bridged.

In a particularly preferred embodiment, the trialkylsilyl-substituted heteroatom-containing organic compound is a compound selected from one of the material classes of phosphines, pyrimidines, pyrazoles, pyrroles, oxazoles, pyrrolidines, imidazoles and/or triazoles.

In an alternative embodiment of the invention, the trialkylsilyl-substituted heteroatom-containing organic compound is an acyclic compound, which allows a coupling via O or N.

### DETAILED DESCRIPTION OF THE INVENTION

One of the most important advantages of the process of the invention is that there is no need to use transition metal-containing catalysts. However, particularly good results are achieved by use of substances from the group of halides of alkali or alkaline earth metals, or an ammonium halide. These substances act as catalysts for the reaction described, so that usually only small quantities are used. Typically, these substances are used in an amount of 0.1 to 100 mol %, particularly in an amount of 5 to 50 mol %, based on the halogen-substituted aromatic. In a preferred embodiment, the reaction thus takes place in the presence of an at least catalytic amount of a halide of the alkali or alkaline earth metals or an ammonium halide. Particularly good results are achieved when the catalytic halide used is a fluoride.

It has been shown that particularly good results are obtained, if the reaction takes place in the presence of an at least catalytic amount of cesium fluoride. In a particularly preferred embodiment, cesium fluoride is used in an amount of 0.1 to 100 mol %, particularly in an amount of 5 to 50 mol %, based on the halogen-substituted aromatic.

While the process of the invention, as discussed further above, enables preparation of aryl-heteroatom-bridged compounds even without the use of transition metal catalysts, the use of transition metal catalysts in a variant of the inventive process can still be beneficial. One embodiment of the inventive process therefore uses transition metal catalysts. Particularly preferred, transition metal catalysts from the group of palladium catalysts are used. It has been shown, that a particular advantage of the inventive process lies in the fact that a very rapid and complete reaction can be achieved, even with the use of a very small amount of transition metal catalyst. In a particularly preferred embodiment, therefore, the process of the invention is carried out with an amount of the transition metal catalyst, for example in the form of a palladium catalyst, in a mole fraction of less than 1 mol %, based on the molar amount of the halogen-substituted aromatic used.

Moreover, it has been shown, that even the use of significantly lower mole fractions of the transition metal catalyst lead to very good results. Therefore, in a particularly preferred embodiment of the process of the invention, the transition metal catalyst, for example in the form of a palladium catalyst, is present in a mole fraction of preferably less than 0.8 mol %, more preferably less than 0.5 mol %, more preferably less than 0.25 mole %, more preferably less than 0.1 mol %, more preferably less than 0.05 mol %, and even more preferably less than 0.01 mol %, each based on the molar amount of halogen-substituted aromatic present.

Within this particularly preferred embodiment, the heteroatom in the aryl-heteroatom-bridged compound is a heteroatom selected from at least one of the group consisting of N, O, S, P.

Another important advantage of the process of the invention is that it can be operated under very mild conditions. In particular, no high temperatures are required to obtain high yields after relatively short reaction times. In a preferred embodiment, the reaction takes place at a temperature in a range of 0 to 120° C., in particular of 40 to 90° C. Often, the process of the invention can even be carried out at room temperature, which further simplifies the process control.

A variety of solvents usable in organic synthesis can be used as solvents for the reaction. Particularly good results are achieved, if the reaction is carried out in a solvent selected from the group comprising DMF (dimethylformamide), DMSO (dimethyl sulfoxide), N-methylpyrrolidine, and THF (tetrahydrofuran).

The invention also relates to the aryl-heteroatom-bridged compounds, which are produced according to the method described above.

Aryl-heteroatom-bridged compounds prepared in this manner represent valuable starting materials for organic synthesis. They are particularly important as starting materials for the synthesis of active ingredients of various kinds. The invention also relates to the use of aryl-heteroatom-bridged compounds prepared according to the process of the invention for the preparation of compounds from the group of pharmaceutical compounds, pesticides and/or herbicides.

In summary, it should be noted that the process of the invention enables synthesis of aryl-heteroatom-bridged compounds in a simpler, faster and cheaper way under mild conditions, and avoiding the use of transition metal-containing catalysts.

### EXAMPLES

The invention described above is explained in more detail below with reference to embodiments:

### Example 1

### Synthesis of 4-imidazol-1-yl-benzonitrile

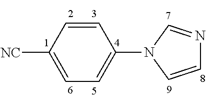

Under a nitrogen atmosphere 362 mg (2.38 mmol) of CsF, previously activated with NaOH, were suspended in 5 ml of DMF and stirred for 30 min. Then, 1.00 g (8.26 mmol) of 4-fluorobenzonitrile was added. After 10 min 1.20 ml (8.18 mmol) N-trimethylsilylimidazole were added and the mixture was stirred at 60° C. for 20 hr. For workup, most of the solvent was removed under vacuum (oil pump), and then 5 ml of water and 5 ml of CH₂Cl₂ were added to the reaction mixture. The organic phase was separated, the aqueous phase was extracted with CH₂Cl₂, the organic phases were combined, washed several times with water and dried over MgSO₄. The solvent was removed under vacuum, and the resulting solid was rinsed twice with pentane.

Yield: 1.07 g (6.31 mmol, 77%), appearance: colorless solid. ¹H NMR (CDCl₃, 25° C., 400.13 MHz): δ=7.25 (s, 1H, ImH), 7.33 (s, 1H, ImH), 7.51-7.53 (m, 2H, PhH), 7.79-7.81 (m, 2H, PhH), 7.94 (s, 1H, ImH). ¹³C NMR (CDCl₃, 25° C., 100.61 MHz): δ=111.3 (C-1), 117.7 (C-9), 117.9 (—CN), 121.5 (C-3, C-5), 131.6 (C-8), 134.2 (C-2, C-6), 135.4 (C-7) 140.6 (C-4).

### Example 2

### Synthesis of 4-pyrrolidine-1-yl-benzonitrile

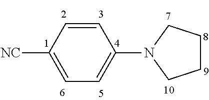

Under a nitrogen atmosphere, 320 mg (2.11 mmol) of CsF were suspended in 5 ml of DMF and stirred for 30 min. Then, 1.00 g (8.26 mmol) of 4-fluorobenzonitrile were added. After 10 min 1.44 ml (8.25 mmol) of N-trimethylsilylpyrrolidine were added, and the mixture was stirred for 6 d at room temperature. For workup, 5 ml of water and 5 ml of CH₂Cl₂ were added to the reaction mixture. The organic phase was separated, the aqueous phase was extracted with CH₂Cl₂, the organic phases were combined, washed several times with water and dried over MgSO₄, and the solvent was removed under vacuum.

Yield: 1.01 g (5.87 mmol, 71%), appearance: pale yellow solid. ¹H NMR (CDCl₃, 25° C., 400.13 MHz): δ=2.00-2.07 (m, 4H, H-8, H-9), 3.30-3.34 (m, 4H, H-7, H-10), 6.47-6.51 (m, 2H, H-3, H-5), 7.42-7.46 (m, 2H, H-2, H-6). ¹³C NMR (CDCl₃, 25° C., 100.61 MHz): δ=25.5 (C-8, C-9), 47.6 (C-7, C-10), 96.7 (C-1), 111.6 (C-3, C-5), 121.1 (—CN), 133.6 (C-2, C-6), 150.1 (C-4).

### Example 3

### Synthesis of 4-(3,5-dimethylpyrazol-1-yl)-benzonitrile

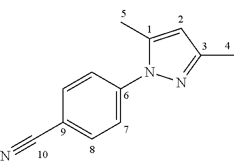

5.0 ml of anhydrous DMF, 0.26 g (2.15 mmol) of 4-fluorobenzonitrile and 0.07 g (0.46 mmol) of CsF were added to 0.36 g (2.14 mmol) of 1-trimethylsilyl-3,5-dimethylpyrazole. The yellow reaction mixture then turns green. After 20 hr, 25 ml of CH₂Cl₂ and 20 ml of water are added, and the aqueous phase is extracted three times with 10 ml each of CH₂Cl₂. The combined organic extracts are washed three times with 25 ml each of water, dried over MgSO₄, and volatile components are removed on a rotary evaporator.

Yield: 0.26 g (1.32 mmol, 62%). C₁₂K₁N₃— ¹H NMR (CDCl₃, 200.13 MHz): δ=2.11 (s, 3H, H-5), 2.23 (d, 3H, H-4), 5.89 (s, 1H, H-2), 7.46 (d, ³J=8.6 Hz, 2H, H-7), 7.58 (d, ³J=8.5 Hz, 2H, H-8) ppm. —¹³C NMR (CDCl₃, 50.32 MHz): δ=12.5 (s, 1C, C-4), 13.0 (s, 1C, C-5), 108.5 (s, 1C, C-2), 109.5 (s, 1C, C-9), 117.9 (s, 1C, C-10), 123.5 (s, 2C, C-7), 132.7 (s, 2C, C-8), 139.3 (s, 1C, C-1), 143.1 (s, 1C, C-6), 150.0 (s, 1C, C-3) ppm.

### Example 4

### Synthesis of 1-(4-nitrophenyl)pyrrolidine

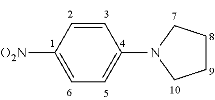

Under a nitrogen atmosphere, 340 mg (2.24 mmol) of CsF, previously activated with NaOH, were suspended in 5 ml of DMF and stirred for 30 min. Then, 1.14 g (8.08 mmol) of 4-fluoronitrobenzene were added. After 10 min 1.44 ml (8.25 mmol) of N-trimethylsilylpyrrolidine were added, and the mixture was stirred at 60° C. for 6 hr. For workup, most of the DMF was removed under vacuum (oil pump) and 15 ml of water and 20 ml of dichloromethane were added to the reaction mixture. The organic phase was separated, the aqueous phase was extracted with dichloromethane, the organic phases were combined, washed several times with water and dried over MgSO₄, and the solvent was removed under vacuum.

Yield: 1.37 g (7.14 mmol, 88%), appearance: orange-colored, crystalline solid. ¹H NMR (CDCl₃, 25° C., 400.13 MHz): δ=2.07-2.11 (m, 4H, H-8, H-9), 3.40-3.44 (m, 4H, H-7, H-10), 6.47-6.50 (m, 2H, H-3, H-5), 8.12-8.15 (m, 2H, H-2, H-6). ¹³C NMR (CDCl₃, 25° C., 100.61 MHz): δ=25.5 (C-8, C-9), 48.0 (C-7, C-10), 110.5 (C-3, C-5), 126.5 (C-2, C-6), 136.6 (C-1), 152.0 (C-4).

### Example 5

### Synthesis of 4-pyrrolidine-1-yl-benzoic acid methyl ester

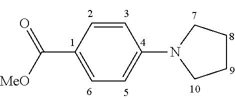

Under a nitrogen atmosphere, 368 mg (2.42 mmol) of CsF, previously activated with sodium hydroxide, were suspended in 5 ml of DMF and stirred for 30 min. Then, 1.25 g (8.11 mmol) of 4-fluorobenzoic acid methyl ester were added. After 10 min 1.44 ml (8.25 mmol) of N-trimethylsilylpyrrolidine were added, and the mixture was stirred at 60° C. for 110 hr. For workup, most of the DMF was removed under vacuum (oil pump), and then 15 ml of water and 25 ml of dichloromethane were added to the reaction mixture. The organic phase was separated, the aqueous phase was extracted with dichloromethane, the organic phases were combined, washed several times with water and dried over magnesium sulfate, and the solvent was removed under vacuum.

Yield: 1.19 g (5.80 mmol, 72%), appearance: colorless crystalline solid. ¹H NMR (CDCl₃, 25° C., 400.13 MHz): δ=2.00-2.03 (m, 4H, H-8, H-9), 3.32-3.35 (m, 4H, H-7, H-10), 6.48-6.51 (m, 2H, H-3, H-5), 7.88-7.90 (m, 2H, H-2, H-6). ¹³C NMR (CDCl₃, 25° C., 100.62 MHz): δ=25.6 (C-8, C-9), 47.7 (C-7, C-10), 51.4 (CH₃), 110.9 (C-3, C-5), 116.8 (C-1), 131.5 (C-2, C-6), 151.1 (C-4), 167.7 (C═O).

### Example 6

### Synthesis of 4-nhenoxybenzonitrile

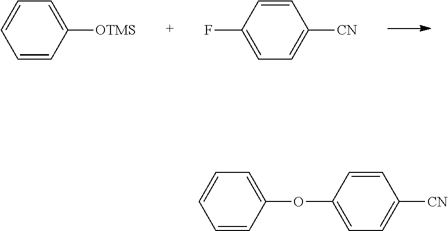

Under a nitrogen atmosphere, 370 mg (2.42 mmol) of CsF, previously activated with sodium hydroxide, were suspended in 5 ml of DMF and stirred for 30 min. Then, 1.01 g (8.34 mmol) of 4-fluorobenzonitrile were added. After 10 min 1.50 ml (8.34 mmol) of phenoxytrimethylsilane were added, and the mixture was stirred at room temperature for 42 hr. For workup, most of the DMF was removed under vacuum (oil pump), and then 15 ml of water and 25 ml of diethyl ether were added to the reaction mixture. The organic phase was separated, the aqueous phase was extracted with diethyl ether, the organic phases were combined, washed several times with water and dried over magnesium sulfate, and the solvent was removed under vacuum. A red oil is obtained, from which the product crystallizes in form of colorless needles.

Yield: 1.27 g (6.51 mmol, 78%), appearance: colorless crystalline solid.

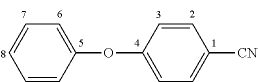

¹H NMR (CDCl₃, 25° C., 400.13 MHz): δ=7.00 (d, J=8.8 Hz, 2H, H-3), 7.06 (d, J=7.9 Hz, 2H, H-6), 7.23 (t, J=7.4 Hz, 1H, H-8), 7.41 (t, J=8.0 Hz, 2H, H-7), 7.59 (d, J=8.8 Hz, 2H, H-2). ¹³C NMR (CDCl₃, 25° C., 100.62 MHz): δ=106.12 (C-1), 118.12 (C-6), 118.77 (CN), 120.41 (C-3), 125.18 (C-8), 130.30 (C-2), 134.18 (C-7), 155.08 (C-4), 161.73 (C-5).

### Example 7

### Synthesis of 4-nitrophenyl propyl ether

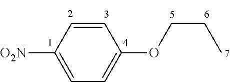

Under a nitrogen atmosphere, 370 mg (2.42 mmol) of CsF, previously activated by evaporation of an aqueous solution (cf. Ph.D. thesis of A. Reis), were suspended in 5 ml of DMF and stirred for 30 min. Then, 1.00 g (7.09 mmol) of 4-fluoronitrobenzene were added. After 10 min 1.00 g (7.55 mmol) of propoxytrimethylsilane were added, and the mixture was stirred at 95° C. for 40 hr. For workup, 30 ml of water and 25 ml of dichloromethane were added to the reaction mixture. The organic phase was separated, the aqueous phase was extracted with dichloromethane, the organic phases were combined, washed several times with water and dried over magnesium sulfate, and the solvent was removed under vacuum. Yield: 0.76 g (4.19 mmol, 59%), red liquid.

¹H NMR (CDCl₃, 25° C., 200.13 MHz) δ=1.05 (t, J=7.4 Hz, 3H, H-7), 1.74-1.94 (m, 2H, H-6), 4.01 (t, J=6.5 Hz, 2H, H-5), 6.86-6.99 (m, 2H, H-3), 8.15-8.22 (m, 2H, H-2). ¹³C NMR (CDCl₃, 25° C., 50.32 MHz): δ=10.49 (C-7), 22.48 (C-6), 70.47 (C-5), 114.54 (C-3), 126.01 (C-2), 141.48 (C-1), 164.40 (C-4).

### Example 8

### Synthesis of Nilandron

### 5,5-Dimethyl (trimethylsilyl)hydantoin

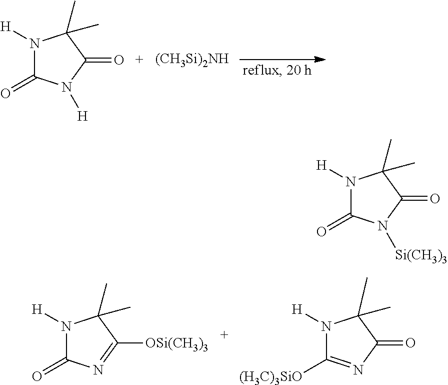

10 g (78.05 mmol) of 5,5-dimethylhydantoin and 12.58 g (78.05 mmol) of hexamethyldisilazane were heated at 80° C. for 20 hr. Excess hexamethyldisilazane is removed under vacuum (oil pump), the crude product which contains several isomeric compounds according to ¹H NMR is used without further purification in the next step of the synthesis. Yield: almost quantitative.

Alternatively: 317 mg (2.40 mmol) of 5,5-dimethylhydantoin and 0.39 g of hexamethyldisilazane (0.50 ml, 2.40 mmol) were reacted together in a microwave oven (automatic power setting of the apparatus) initially for 5 min at 100° C., followed by an additional 15 min at 120° C. Volatile components were removed under vacuum (oil pump). Yield: 0.33 g (1.65 mmol, 69%), colorless solid (mixture of isomers).

### Nilandron:

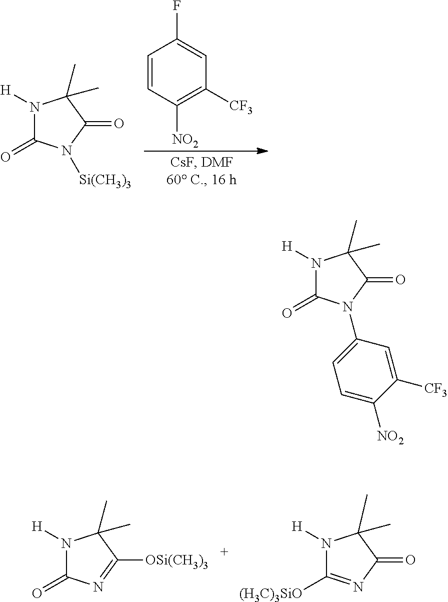

Under a nitrogen atmosphere 128 mg (0.84 mmol) of CsF, previously activated with sodium hydroxide, were suspended in 3 ml of DMF and stirred at room temperature for 30 min. Then, 0.74 g (3.49 mmol) of 4-nitro-2-trifluoromethylfluorobenzene are added. After 10 min 0.70 g (3.49 mmol) of 5,5-dimethyl(trimethylsilyl)hydantoin (mixture of isomers) are added, and the mixture is stirred at 60° C. for 16 hr. For workup, most of the DMF is removed under vacuum (oil pump), and 15 ml of water and 25 ml of diethyl ether are added to the reaction mixture. The organic phase is separated, the aqueous phase is extracted with diethyl ether, the organic phases are combined, washed several times with water and dried over magnesium sulfate, and the solvent is removed under vacuum. A yellow oil is obtained.

Yield: 0.80 g. C₁₂H₁₀F₃N₃O₄: calcd. C, 45.43; H, 3.18; N, 13.25. found: C, 45.21; H, 3.41; N, 12.69. ¹H NMR (d₆-DMSO, 600 MHz): δ=1.43 (s, 6H, CH₃), 8.07 (dd, 1H, ³J=8.6 Hz, ⁵J=2.0 Hz, 6-H), 8.21 (d, 1H, ⁴J=1.8 Hz, 3-H), 8.31 (d, 1H, ³J=8.8 Hz, 5-H), 8.84 (s, 1H, NH). ¹³C NMR (d₆-DMSO, 150 MHz): δ=24.6 (CH₃), 58.1 (C-9), 121.9 (q, ¹J=273.3 Hz, CF₃), 121.9 (q, ²J=33.3 Hz, C-2), 125.3 (q, ³J=5.6 Hz, C-3), 126.5 (C-5), 131.1 (C-6), 136.6 (C-4), 145.2 (C-1), 153.1 (C-7), 175.8 (C-8). ¹⁹F NMR (d₆-DMSO, 565 MHz): δ=−59.0 (CF₃).

### Example 9

### 2-(3-N,N-dimethylamino-prop-2-en-1-onyl)phenyldiphenylphosphine

Under a nitrogen atmosphere 700 ml of anhydrous DMF, 171.58 g (888.00 mmol) of 1-(2-fluorophenyl)-3-(N,N-dimethylamino)prop-2-ene-1-one and 117.94 g (776.32 mmol) of CsF are added to a 2 L three-necked flask with nitrogen connection and dripping funnel and a cooling trap (ice cooling). To this mixture 237.00 ml (889.05 mmol) of diphenyl(trimethylsilyl)phosphine are added dropwise at a rate of approximately 1 drop per second. After completion of the addition, stirring is continued for 48 hr under inert gas. Then, 800 ml of CH₂Cl₂ and 700 ml of water are added, the phases are mixed vigorously, and after phase separation the aqueous phase is decanted. The aqueous phase is extracted with 200 ml of CH₂Cl₂, the organic phase is washed with 400 ml of water and the combined organic phases are dried over MgSO₄. After evaporation of the solvent a pale yellow solid remains, which is rinsed with 400 ml of diethyl ether. After drying 224.55 g (70.28% of theoretical) of the product are obtained.

Analysis calculated for C₂₃H₂₂NOP: C, 76.86; H, 6.17; N, 3.90. Found: C, 76.30; H, 6.22; N, 3.95%. IR (KBr, cm⁻¹): 3416 w, 3048 w, 2994 w, 1637 s, 1568 s, 1547 s, 1431 s, 1354 s, 1273 m, 1030 m, 898 m, 750 s, 696 s, 504 m. ¹H NMR (400.1 MHz, 25° C., CDCl₃): δ=7.63 (m, 1H, H_{Ar}), 7.37 (t, 1H, H_{Ar}), 7.26-7.35 (m, 12H, H_{Ar}), 7.05 (dd, ³J_{Hp}=3.0 Hz, ³J_{HH}=7.7H, 1H, H_{Ar}), 5.41 (d, 1H, ³J_{HH}=12.6 Hz, CO—CH), 2.94, 2.69 (2×s, 6H, CH₃). ¹³C NMR (100.61 MHz, 25° C., CDCl₃): δ=191.7 (s, CO) 154.8 (s), 147.9 (d, ¹J_{cp}=26.8 Hz), 139.1 (d, ²J_{cp}=12.0 Hz), 136.2 (d, ¹J_{cp}=19.4 Hz), 134.8 (s), 133.9 (d, ²J_{cp}=20.3 Hz), 129.3 (s), 128.5 (s), 128.4 (d, ³J_{cp}=6.4 Hz), 128.3 (s), 127.6 (d, ³J_{cp}=5.5 Hz), 96.9 (s), 44.9, 37.2 (2×s, CH₃). ³¹P NMR (161.98 MHz, 25° C., CDCl₃): δ=−8.95 (s) ppm.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-11. (canceled)

12. A process for preparing aryl-heteroatom-bridged compounds having an aryl residue bound directly to a hetero atom which is part of a heteroatom-containing organic residue, the method comprising reacting a halogen-substituted aromatic compound with a trialkylsilyl-substituted heteroatom-containing organic compound, wherein the trialkylsilyl-substituted heteroatom-containing organic compound is a compound selected from at least one of the classes of phosphines, pyrimidines, pyrazoles, pyrroles, oxazoles, pyrrolidines, imidazoles, and triazoles.

13. The process according to claim 12, wherein the halogen-substituted aromatic compound comprises a mono- or di-halogen-substituted aromatic compound.

14. The process according to claim 12, wherein the halogen-substituted aromatic compound comprises a fluorine-substituted aromatic compound.

15. The process according to claim 12, wherein the trialkylsilyl-substituted heteroatom-containing organic compound comprises a trimethylsilyl-substituted heteroatom-containing organic compound.

16. The process according to claim 12, wherein the reaction takes place in the presence of at least a catalytic amount of a halide selected from alkali metal halides, alkaline earth metal halides and ammonium halide.

17. The process according to claim 16, wherein the halide is a fluoride.

18. The process according to claim 16, wherein the reaction takes place in the presence of at least a catalytic amount of cesium fluoride.

19. The process according to claim 12, wherein the reaction takes place in the presence of a transition metal catalyst having a mole fraction of less than 1 mol %, based on the molar amount of the halogen-substituted aromatic compound.

20. The process according to claim 12, wherein the heteroatom comprises at least one atom selected from the group of N, O, S, and P.

21. The process according to claim 12, wherein the reaction takes place at a temperature in a range of 0 to 120° C.

22. The process according to claim 12, wherein the reaction takes place at a temperature in a range of 40 to 90° C.

23. The process according to claim 12, wherein the reaction takes place in a solvent selected from the group of DMF, DMSO, THF, and N-methylpyrrolidine.
